(19) United States
(12) Patent Application Publication
(10) Pub. No.: US 2003/0149364 A1
(43) Pub. Date: Aug. 7, 2003

(54) Methods, system and apparatus for digital imaging

(76) Inventors: Ajay Kapur, Clifton Park, NY (US); Jeffrey Wayne Eberhard, Albany, NY (US); Boris Yamrom, Bronx, NY (US); Kai E. Thomenius, Clifton Park, NY (US); Donald Joseph Buckley JR., Schenectady, NY (US); Roger Neal Johnson, Hagaman, NY (US); Reinhold F. Wirth, Ballston Spa, NY (US); Oliver Astley, Clifton Park, NY (US); Beale Opsahl-Ong, Darien, CT (US); Serge Louis Wilfrid Muller, Guyancourt (FR); Steve Karr, Scotia, NY (US)

Correspondence Address: John S. Beulick, Armstrong Teasdale LLP, Suite 2600, One Metropolitan Sq., St. Louis, MO, 63102, US

(21) Appl. No.: 10/062,334

(22) Filed: Feb. 1, 2002

(51) Int. Cl. A61B 5/05; A61B 8/00; A61B 8/12; A61B 8/14

(52) U.S. Cl. 600/439

(57) ABSTRACT

A method for generating an image of an object of interest includes acquiring a first three-dimensional dataset of the object at a first position using an X-ray source and a detector, acquiring a second three-dimensional dataset of the object at the first position using an ultrasound probe, and combining the first three-dimensional dataset and the second three-dimensional dataset to generate a three-dimensional image of the object.

### STATEMENT REGARDING FEDERALLY SPONSORED RESEARCH & DEVELOPMENT

The government may have rights in this invention pursuant to Subcontract 22287 issued from the Office of Naval Research/Henry M. Jackson Foundation.

### BACKGROUND OF THE INVENTION

This invention relates generally to digital imaging and more particularly to a method, system, and apparatus for acquiring digital images using an X-ray source and detector, and an ultrasound device.

In at least some known imaging systems, a radiation source projects a cone-shaped beam which passes through the object being imaged, such as a patient and impinges upon a rectangular array of radiation detectors. In at least one known tomosynthesis system, the radiation source rotates with a gantry around a pivot point, and views of the object may be acquired for different projection angles. As used herein “view” refers to a single projection image or, more particularly, “view” refers to a single projection radiograph which forms a projection image. Also, as used herein, a single reconstructed (cross-sectional) image, representative of the structures within the imaged object at a fixed height above the detector, is referred to as a “slice”. And a collection, or plurality, of views is referred to as a “projection dataset.” A collection of, or a plurality of, slices for all heights is referred to as a “three-dimensional (3D) dataset representative of the image object.”

In other known medical imaging systems, ultrasound diagnostic equipment is used to view organs of a subject. Conventional ultrasound diagnostic equipment typically includes an ultrasound probe for transmitting ultrasound signals into the subject and receiving reflected ultrasound signals therefrom. The reflected ultrasound signals received by the ultrasound probe are processed and an image of the target under examination is formed.

Conventional breast imaging is based on standard 2D X-ray mammography for screening, and X-ray and ultrasound for diagnostic follow-up. Ultrasound is particularly effective at differentiating benign cysts and masses, and X-ray is typically used for detailed characterization of microcalcifications. Combining the images generated using the X-ray and detector and the images generated using the ultrasound system may leverage the strengths of both modalities, however registration of the images is challenging since the X-ray examination is typically accomplished with the breast compressed and the ultrasound examination is typically performed by scanning an uncompressed breast. Additionally, the ultrasound scan is typically done manually, which increases the variability of the results and the difficulty in registering the results.

### BRIEF DESCRIPTION OF THE INVENTION

In one aspect, a method for generating an image of an object of interest is provided. The method includes acquiring a first three-dimensional dataset of the object at a first position using an X-ray source and a detector, acquiring a second three-dimensional dataset of the object at the first position using an ultrasound probe, and combining the first three-dimensional dataset and the second three-dimensional dataset to generate a three-dimensional image of the object.

In another aspect, a method for generating an image of an object of interest is provided. The method includes compressing an object of interest using a compression paddle, acquiring a first three-dimensional dataset of the object at a first position using an X-ray source and a detector, and positioning an ultrasound probe mover assembly adjacent the compression paddle such that the second three-dimensional dataset obtained with the ultrasound probe mover assembly is co-registered with the first three-dimensional dataset obtained through the compression paddle by mechanical design. The method also includes coupling an ultrasound probe with the probe mover assembly such that the ultrasound probe emits an ultrasound output signal through the compression paddle and the object of interest, acquiring a second three-dimensional dataset of the object at the first position using an ultrasound probe, and combining the first three-dimensional dataset and the second three-dimensional dataset to generate a three-dimensional image of the object.

In still another aspect, a method for generating an image of an object of interest is provided. The method includes compressing an object of interest using a compression paddle, acquiring a two-dimensional dataset of the object, at a first position, using an X-ray source and a detector, and positioning an ultrasound probe mover assembly adjacent the compression paddle such that the second three-dimensional dataset obtained with the ultrasound probe mover assembly is co-registered with the first three-dimensional dataset obtained through the compression paddle by mechanical design. The method also includes operationally coupling an ultrasound probe with the probe mover assembly such that the ultrasound probe emits an ultrasound output signal through the compression paddle and the object of interest, acquiring a three-dimensional dataset of the object, at the first position, using an ultrasound probe, and combining the two-dimensional dataset and the second three-dimensional dataset to generate a three-dimensional image of the object.

In a further aspect, an apparatus is provided. The apparatus includes a compression paddle, an ultrasound probe mover assembly mechanically aligned with the compression paddle, and an ultrasound probe coupled with the probe mover assembly such that the ultrasound probe emits an ultrasound output signal through the compression paddle and the object of interest.

In a still further aspect, a medical imaging system for generating an image of an object of interest is provided. The medical imaging system includes a detector array, at least one radiation source, a compression paddle, an ultrasound probe mover assembly mechanically aligned with the compression paddle, an ultrasound probe coupled with the probe mover assembly such that the ultrasound probe emits an ultrasound output signal through the compression paddle and the object of interest, and a computer coupled to the detector array, the radiation source, and the ultrasound probe. The computer is configured to acquire a first three-dimensional dataset of the object at a first position using the X-ray source and the detector, acquire a second three-dimensional dataset of the object at the first position using the ultrasound probe, and combine the first three-dimensional dataset and the second three-dimensional dataset to generate a three-dimensional image of the object.

In a further aspect, a compression paddle is provided. The paddle includes a plurality of composite layers. The layers are sonolucent and radiolucent

### BRIEF DESCRIPTION OF THE DRAWINGS

FIG. 1 is a pictorial view of an imaging system.

FIG. 2 is a flow diagram of a method for generating an image of an object of interest.

FIG. 3 is a side view of a portion of a novel compression paddle.

FIG. 4 is a top view of probe mover assembly.

FIG. 5 is a flow diagram of an exemplary method for generating an image of an object.

FIG. 6 a pictorial view of a medical imaging system.

FIG. 7 is a pictorial view of a compression paddle system and interface and ultrasound imaging system.

FIG. 8 is a side view of a portion of a medical imaging system shown in FIG. 1.

FIG. 9 is an image illustrating exemplary effects of refractive corrections.

FIG. 10 is the same image illustrated in FIG. 9 without the refractive corrections.

### DETAILED DESCRIPTION OF THE INVENTION

FIG. 1 is a pictorial view of a medical imaging system 12. In an exemplary embodiment, imaging system 12 includes an ultrasound imaging system 14, a probe mover assembly 16, an ultrasound probe 18, and at least one of an x-ray imaging system and a tomosynthesis imaging system 20. In the exemplary embodiment, ultrasound imaging system 14, probe mover assembly 16, ultrasound probe 18, and tomosynthesis imaging system 20 are operationally integrated in imaging system 12. In another embodiment, ultrasound imaging system 14, probe mover assembly 16, ultrasound probe 18, and tomosynthesis imaging system 20 are physically integrated in a unitary imaging system 12.

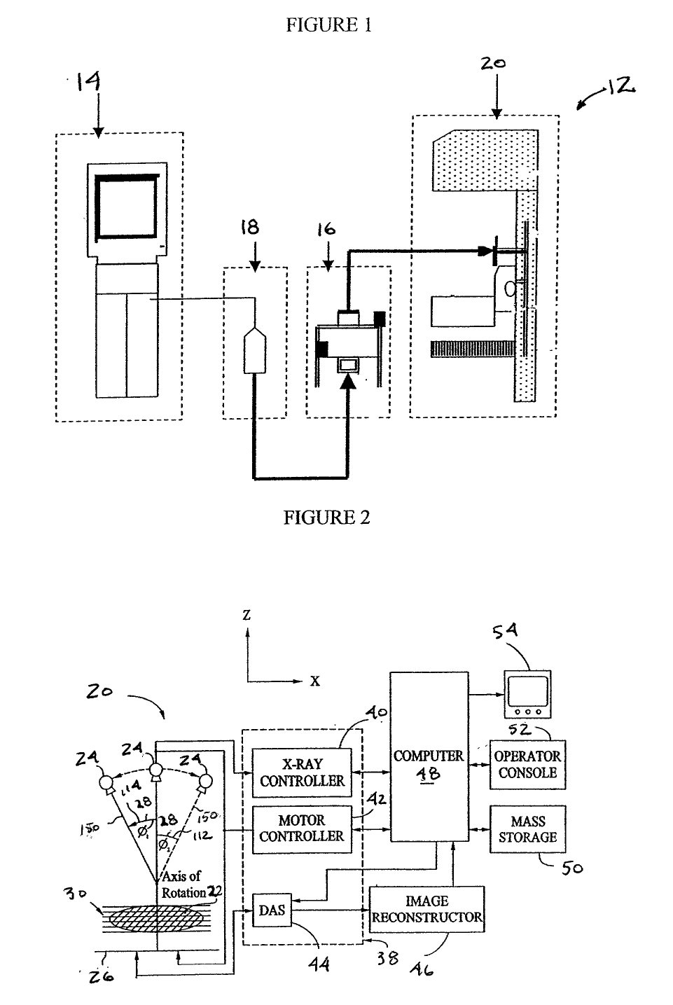

FIG. 2 is a pictorial view of tomosynthesis imaging system 20. In the exemplary embodiment, tomosynthesis imaging system 20 is used to generate a three-dimensional dataset representative of an imaged object 22, such as a patient's breast. System 20 includes a radiation source 24, such as an X-ray source, and at least one detector array 26 for collecting views from a plurality of projection angles 28. Specifically, system 20 includes a radiation source 24 which projects a cone-shaped beam of X-rays which pass through object 22 and impinge on detector array 26. The views obtained at each angle 28 may be used to reconstruct a plurality of slices, i.e., images representative of structures located in planes 30 which are parallel to detector 26. Detector array 26 is fabricated in a panel configuration having a plurality of pixels (not shown) arranged in rows and columns, such that an image is generated for an entire object 22 of interest, such as a breast.

Each pixel includes a photosensor, such as a photodiode (not shown), that is coupled via a switching transistor (not shown) to two separate address lines (not shown). In one embodiment, the two lines are a scan line and a data line. The radiation incident on a scintillator material and the pixel photosensors measure, by way of change in the charge across the diode, an amount of light generated by X-ray interaction with the scintillator. More specifically, each pixel produces an electronic signal that represents an intensity, after attenuation by object 22, of an X-ray beam impinging on detector array 26. In one embodiment, detector array 26 is approximately 19 centimeters (cm) by 23 cm and is configured to produce views for an entire object 22 of interest, e.g., a breast. Alternatively, detector array 26 is variably sized depending on the intended use. Additionally, a size of the individual pixels on detector array 26 is selected based on the intended use of detector array 26.

In the exemplary embodiment, the reconstructed three-dimensional dataset is not necessarily arranged in slices corresponding to planes that are parallel to detector 26, but in a more general fashion. In another embodiment, the reconstructed dataset consists only of a single two-dimensional image, or one-dimensional function. In a further embodiment, detector 26 is a shape other than planar.

In the exemplary embodiment, radiation source 24 is moveable relative to object 22. More specifically, radiation source 24 is translatable such that the projection angle 28 of the imaged volume is altered. Radiation source 24 is translatable such that projection angle 28 may be any acute or oblique projection angle.

The operation of radiation source 24 is governed by a control mechanism 38 of imaging system 20. Control mechanism 38 includes a radiation controller 40 that provides power and timing signals to radiation source 24, and a motor controller 42 that controls a respective translation speed and position of radiation source 24 and detector array 26. A data acquisition system (DAS) 44 in control mechanism 38 samples digital data from detector 26 for subsequent processing. An image reconstructor 46 receives sampled and digitized projection dataset from DAS 44 and performs high-speed image reconstruction, as described herein. The reconstructed three-dimensional dataset, representative of imaged object 22, is applied as an input to a computer 48 which stores the three-dimensional dataset in a mass storage device 50. Image reconstructor 46 is programmed to perform functions described herein, and, as used herein, the term image reconstructor refers to computers, processors, microcontrollers, microcomputers, programmable logic controllers, application specific integrated circuits, and other programmable circuits.

Computer 48 also receives commands and scanning parameters from an operator via a console 52 having an input device. A display 54, such as a cathode ray tube and a liquid crystal display (LCD), allows the operator to observe the reconstructed three-dimensional dataset and other data from computer 48. The operator supplied commands and parameters are used by computer 48 to provide control signals and information to DAS 44, motor controller 42, and radiation controller 40.

Imaging system 20 also includes a compression paddle 56 that is positioned adjacent probe mover assembly 16 such probe mover assembly 16 and compression paddle 56 are mechanically aligned. Further, an ultrasound dataset, i.e. a second three-dimensional dataset, obtained with probe mover assembly 16 is co-registered with an x-ray dataset, i.e. a first three-dimensional dataset, obtained through compression paddle 56 by mechanical design. In one embodiment, ultrasound probe 18 is operationally coupled with probe mover assembly 16 such that ultrasound probe 18 emits an ultrasound output signal through compression paddle 56 and breast 22, which is at least partially reflected when an interface, such as a cyst, is encountered within breast 22. In another embodiment, ultrasound probe 18 is a 2D array of capacitative micro-machined ultrasonic transducers that are operationally coupled to compression paddle 56, and probe mover assembly 16 is not used.

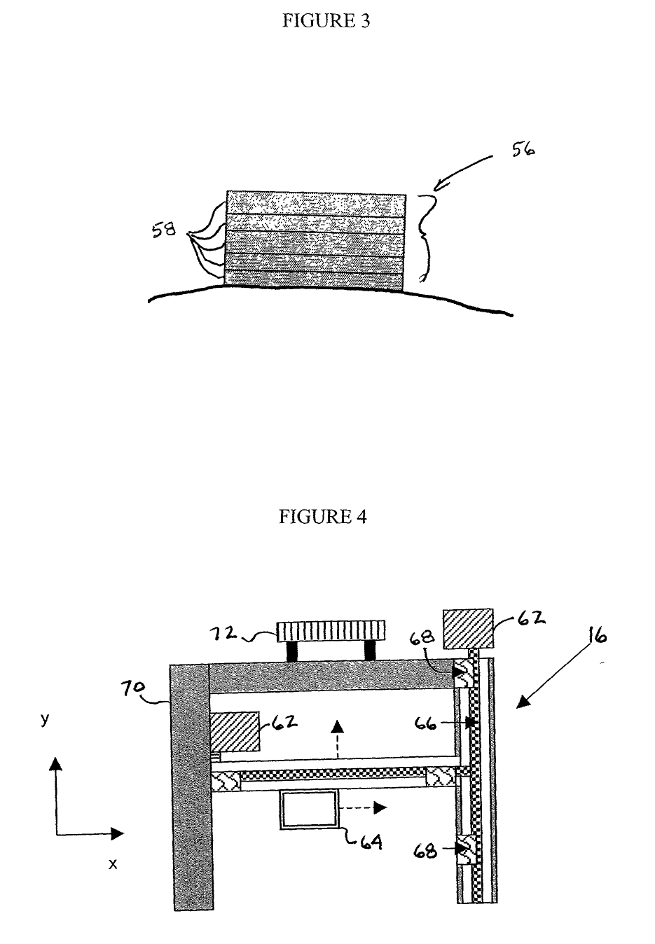

FIG. 3 is a side view of compression paddle 56. In one embodiment, compression paddle 56 is acoustically transparent (sonolucent) and X-ray transparent (radiolucent), and fabricated from a composite of plastic materials, such as, but not limited to materials listed in Table 1, such that an attenuation coefficient of compression paddle 56 is less than approximately 5.0 decibels per centimeter when system 2 is operating at approximately 10 megahertz, thereby minimizing ultrasonic reverberations and attenuation through compression paddle 58. In another embodiment, compression paddle 56 is fabricated using a single composite material. In a further embodiment, compression paddle 56 is fabricated using a single non-composite material. In the exemplary embodiment, compression paddle 56 is approximately 2.7 millimeters (mm) in thickness and includes a plurality of layers 58. Layers 58 are fabricated using a plurality of rigid composite materials, such as, but not limited to polycarbonates, polymethylpentenes, and polystyrenes. Compression paddle 56 is designed using a plurality of design parameters shown in Table 1. Compression paddle 56 design parameters include, but are not limited to, an X-ray attenuation, an atomic number, an optical transmission, a tensile modulus, a speed of sound, a density, an elongation, a Poisson ratio, an acoustic impedance, and an ultrasonic attenuation.

TABLE 1

| | | Acoustic | | | | X-Ray | | Optical | Mechanical | | |
|---|---|---|---|---|---|---|---|---|---|---|---|
| | | | | | Attenuation @ | | Color | Trans- | Tensile | | |
| | | Density | speed | impe- | 5 MHz | Attenuation | Change | mission | Modulus | Elongation | Poisson |
| Material | Acronym | g/cm³ | mm/μs | dance | dB/cm | % in 3 mm | 10 = none | % | (GPA) | % | Ratio |
| Polymethylpentene | PMP, TP | 0.83 | 2.22 | 1.84 | 4.6 | 9.4 | 8 | 80 | 1.5 | 17 | 0.33 |
| Polycarbonate | PC | 1.18 | 2.27 | 2.68 | 23.2 | 14.8 | 5 | 90 | 2.1 | 40 | 0.33 |
| Polystyrene | PS | 1.05 | 2.4 | 2.52 | 1.8 | 14.7 | 9 | 90 | 2.38 | 2 | 0.33 |
| Polyethylene Tere- | PET | 1.37 | 2.54 | 3.48 | 5 | 15.6 | 2 | 100 | 3.2 | 5 | 0.33 |
| phthalate | | | | | | | | | | | |
| Epoxy | | 1.21 | 2.8 | 3.39 | 6 | 52.2 | 8 | 80 | 14.7 | 5 | 0.33 |
| Polysulfone | PSF | 1.24 | 2.24 | 2.78 | 10.6 | 56.9 | 5 | 80 | 2.6 | 35 | 0.33 |
| Polyethylene (low den- | PE | 0.91 | 1.95 | 1.77 | 2.4 | 10.7 | 9 | 10 | 1.05 | 10 | 0.33 |
| sity) | | | | | | | | | | | |
| Polymethylmethacryl- | PMMA | 1.19 | 2.75 | 3.27 | 6.4 | 14.8 | 5 | 92 | 3.1 | 2 | 0.33 |
| ate | | | | | | | | | | | |
| Polypropylene | PP | 0.88 | 2.74 | 2.41 | 5.1 | 10.7 | 9 | 10 | 1.05 | 10 | 0.33 |
| Polyvinyl Chloride | PVC | 1.15 | 2.33 | 2.68 | 12.8 | 64.4 | 0 | 85 | 0.004 | 440 | 0.33 |
| Silicone Rubber | SR | 1.05 | 1.05 | 1.10 | 24 | 37.9 | 10 | 25 | 0.003 | 200 | 0.50 |
| Styrene Butadiene | SBR | 1.02 | 1.92 | 1.96 | 24.3 | 20.1 | 2 | 25 | 0.003 | 200 | 0.50 |
| Rubber | | | | | | | | | | | |

Fabricating compression paddle 56 using a plurality of composite layers 58, facilitates, an effective X-ray attenuation coefficient and point spread function that is similar to that of polycarbonate for mammographic spectra. Additionally, an optical transmission greater than 80%, a low ultrasonic attenuation (less than 3 dB) at ultrasound probe frequencies up to approximately 12 megahertz. (MHz) may be achieved using composite layers 58. Further, composite layers 58 facilitate a maximum intensity of interface reflections within 2% of a maximum beam intensity, less than 1 mm deflection from the horizontal over a 19×23-cm² area exposed to a total compression force of 18 daN, and a mechanical rigidity and a plurality of radiation resistance properties over time similar to polycarbonate.

FIG. 4 is a top view of probe mover assembly 16. In one embodiment, probe mover assembly 16 is removably coupled to paddle 56 and may be de-coupled from compression paddle 56, such that probe mover assembly 16 may be positioned independently above compression paddle 56. Probe mover assembly 16 includes a plurality of stepper motors 62, a position encoder (not shown) and a plurality of limit switch driven carriages (not shown), which includes at least one carriage which mounts ultrasound probe 18 (shown in FIG. 1) through a receptacle 64 to enable variable vertical positioning capabilities of compression paddle 56. In one embodiment, ultrasound probe 18 descends vertically in a z-direction until contact is made with compression paddle 56. Stepper motors 62 drive ultrasound probe 18 along carriages 66 in fine increments in the x and y directions using a variable speed determined by a user. Limit switches 68, along with backlash control nuts (not shown), facilitate preventing ultrasound probe 18 from moving beyond a pre-determined mechanical design of probe mover assembly 16 limits. Ultrasound probe 18 is mounted on a U-shaped plate 70 that is attached to a receptacle 72. In one embodiment, U-shaped plate 70 attaches to a plurality of guide rails (not shown) on the x-ray imaging system or tomosynthesis imaging system 20 through a separate assembly (not shown). Probe mover assembly 16 dimensions, in the x and y directions, are variably selected based on a desired range of ultrasound probe 18 motion compared to the dimensions of compression paddle 56. In the z direction the dimensions are limited by a vertical clearance between radiation source 24 housing above probe mover assembly 16 and compression paddle 56 below it.

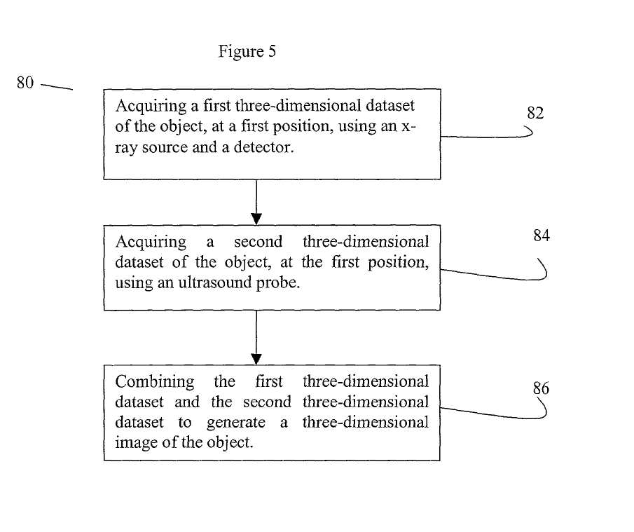

FIG. 5 is a flow diagram of an exemplary method 80 for generating an image of an object 22 of interest. Method 80 includes acquiring 82 a first three-dimensional dataset of object 22, at a first position, using X-ray source 24 and detector 26, acquiring 84 a second three-dimensional dataset of object 22, at the first position, using an ultrasound probe 18, and combining 86 the first three-dimensional dataset and the second three-dimensional dataset to generate a three-dimensional image of object 22.

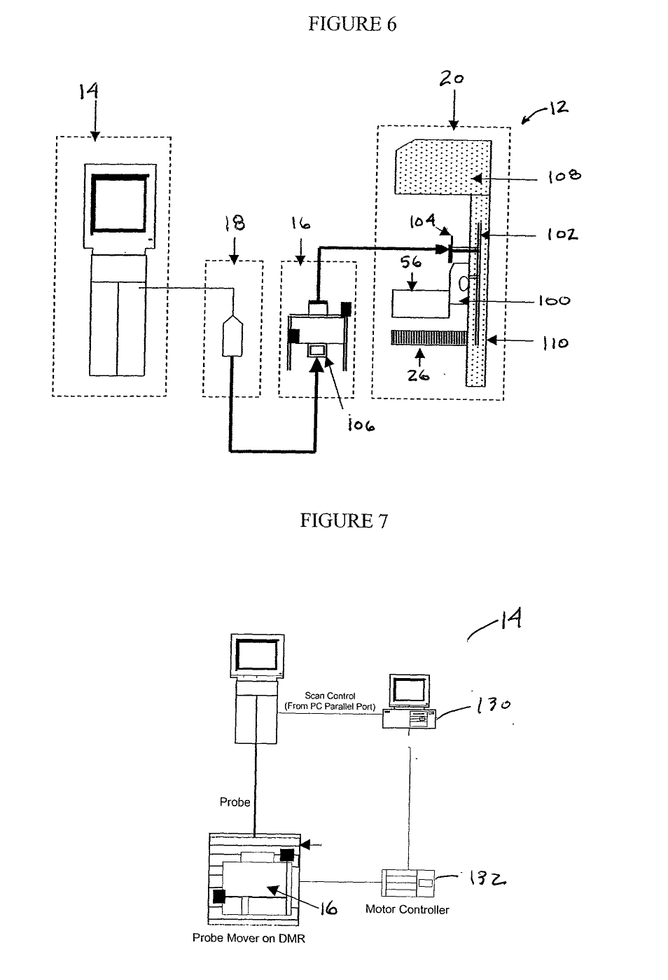

FIG. 6 a pictorial view of imaging system 12. In use, and referring to FIG. 6, compression paddle 56 is installed in tomosynthesis imaging system 20 through a compression paddle receptacle 100. In one embodiment, probe mover assembly 16 is attached to a receptacle (not shown) on a plurality of guide rails (not shown) on an X-ray positioner 102, above a compression paddle receptacle (not shown) through an attachment 104. In another embodiment, probe mover assembly 16 is attached using a plurality of side handrails (not shown) on tomosynthesis imaging system 20. Ultrasound probe 18 is connected to the ultrasound imaging system 14 on one end, and interfaces with probe mover assembly 16 through a probe receptacle 106. A patient is placed adjacent tomosynthesis imaging system 20 such that breast 22 is positioned between compression paddle 56 and detector 26.

Ultrasound probe 18 and probe mover assembly 16 geometry are calibrated with respect to compression paddle 56. In one embodiment, calibrating ultrasound probe 18 includes ensuring that ultrasound probe 18 is installed into probe mover receptacle 104, and probe mover assembly 16 is attached to tomosynthesis imaging system 20 through compression paddle receptacle 100. Calibrating imaging system 12 facilitates ensuring that the transformation operations between co-ordinate systems is validated. A correct beam-forming code environment is installed on ultrasound imaging system 14 to facilitate correcting refractive effects through compression paddle 56. Optimal parameters are then determined based on a prior knowledge of the patient or previous X-ray or ultrasound examinations.

The patient is positioned in at least one of a cranio-caudal, medial-lateral, and an oblique position, such that breast 22, or object 22 of interest, is positioned between compression paddle 56 and detector 26. In one embodiment, breast 22 is slightly covered with a lubricant, such as, but not limited to, a mineral oil. Compression paddle 56 is then used to compress breast 22 to an appropriate thickness using at least one of a manual control on receptacle 100 and an automatic control for receptacle 100.

An X-ray examination is then taken with tomosynthesis imaging system 20 operating in at least one of a standard 2D and a tomosynthesis mode. In the tomosynthesis mode, an X-ray tube housing 108 is modified to enable rotational capabilities about an axis vertically above detector 26 independent of a positioner 110. In one embodiment, the patient and detector 26 are fixed, and tube housing 108 rotates.

Views of breast 22, are then acquired from at least two projection angles 28 (shown in FIG. 2) to generate a projection dataset of the volume of interest. The plurality of views represent the tomosynthesis projection dataset. The collected projection dataset is then utilized to generate a first three-dimensional dataset, i.e., a plurality of slices for scanned breast 22, that is representative of the three-dimensional radiographic representation of imaged breast 22. After enabling radiation source 24 such that the radiation beam is emitted at a first projection angle 112 (shown in FIG. 2), a view is collected using detector array 26. Projection angle 28 of system 20 is then altered by translating the position of source 24 such that central axis 150 (shown in FIG. 2) of the radiation beam is altered to a second projection angle 114 (shown in FIG. 2) and such that a position of detector array 26 is altered to facilitate breast 22 remaining within the field of view of system 20. Radiation source 24 is again enabled and a view is collected for second projection angle 114. The same procedure is then repeated for any number of subsequent projection angles 28.

In one embodiment, a plurality of views of breast 22 are acquired using radiation source 24 and detector array 26 at a plurality of angles 28 to generate a projection dataset of the volume of interest. In another embodiment, a single view of breast 22 is acquired using radiation source 24 and detector array 26 at an angle 28 to generate a projection dataset of the volume of interest. The collected projection dataset is then utilized to generate at least one of a 2D dataset and a first 3D dataset for scanned breast 22. The resultant data are stored in a designated directory on computer 38 (shown in FIG. 2). If tomosynthesis scans are taken, the gantry should be returned to its vertical position.

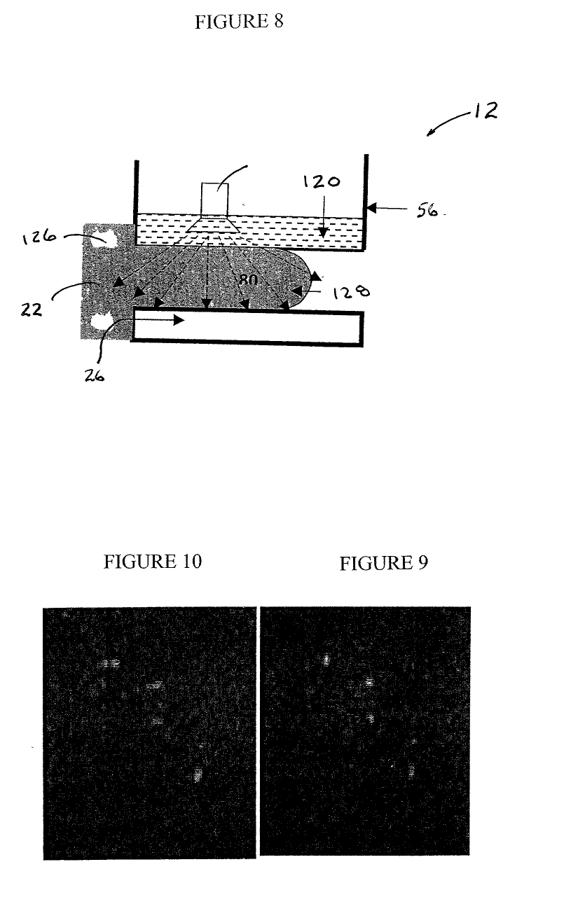

FIG. 7 is a pictorial view of compression paddle 56 and an interface between ultrasound imaging system 14 and tomosynthesis imaging system 20. FIG. 8 is a side view of a portion of imaging system 12. In the exemplary embodiment, compression paddle 56 is filled with acoustic coupling gel 120 to approximately 2 mm height above compression paddle 56. In another embodiment, an acoustic sheath (not shown) is positioned on compression paddle 56. Probe mover assembly 16 is attached to tomosynthesis imaging system 20 gantry (not shown) through attachment 104 (shown in FIG. 6) such that a probe mover assembly plane is parallel to a plane of compression paddle 56. In one embodiment, ultrasound probe 18 is lowered until the acoustic sheath is contacted. In another embodiment, ultrasound probe 18 is lowered until partially immersed in coupling gel 120. Ultrasound probe 18 height is adjusted through receptacle 106 (shown in FIG. 6).

Ultrasound probe 18, vertically mounted above compression paddle 56, is electro-mechanically scanned over entire breast 22 including chest wall 126 and nipple regions 128, to generate a second 3D dataset of breast 22. In one embodiment, a computer 130 drives a stepper controller 132 to scan breast 22 in a rastor-like fashion. In another embodiment, computer 38 (shown in FIG. 2) drives a controller 132 to scan breast 22 in a rastor-like fashion. At least one of computer 38 and computer 130 includes software which includes electronic beam steering and elevation focusing capabilities. In one embodiment real time ultrasound data may be viewed on a monitor of ultrasound imaging system 14. In another embodiment, ultrasound data may be viewed on any display, such as but not limited to display 54 (shown in FIG. 2). Probe mover assembly 16 is removed from tomosynthesis imaging 20, and compression paddle 56 is repositioned to release the patient.

Electronic beam steering enables the chest wall and nipple regions to be imaged as shown in FIG. 8 by looking for example at nipple region 128. If ultrasound probe 18 is directly over nipple region 128, the air gaps between compressed breast 22 and compression paddle 56 would not let the acoustic energy be transferred to nipple region 128. However with the steered beams shown entering from the left in FIG. 8, the acoustic energy is efficiently transferred, thereby reducing the need to place conforming gel pads to allow nipple region 128 to be imaged. Further beam steering may be controlled such that acoustic shadowing due to structures such as Cooper's ligaments may be minimized by steering the beam at a number of angles and then compounding the data sets.

In one embodiment, the co-ordinate system of the first dataset is transformed into that of the second dataset, thereby allowing the datasets to be registered by hardware design and registration corrected for intermittent patient motion using imaged based registration methods. Alternatively, the co-ordinate system of the second dataset is transformed into that of the first dataset. Since the first 3D dataset and the second 3D dataset are acquired in the same physical configuration of breast 22, the images may be registered directly from the mechanical registration information. Specifically, the images may be registered directly on a point by point basis throughout the breast anatomy, thereby eliminating ambiguities associated with registration of 3D ultrasound images with 2D X-ray images. Alternately, the physics of the individual imaging modalities may be used to enhance the registration of the two images. Differences in spatial resolution in the two modalities, and in propagation characteristics may be taken into account to identify small positioning differences in the two images. Registration is then based on corrected positions in the 3D data sets. Matching regions of interest on either image dataset may then be simultaneously viewed in a plurality of ways, thereby enhancing qualitative visualization and quantitative characterization of enclosed objects or local regions.

FIG. 9 is an image illustrating exemplary effects of refractive corrections at 12 MHz. FIG. 10 is the same image illustrated in FIG. 9 without the refractive corrections. In one embodiment, refractive corrections from compression paddle 56 are in built into the beam forming process as shown in FIGS. 9 and 10. The diffuse appearance of the wires is corrected for with the refraction corrections for a 3 mm plastic material. In one embodiment, ultrasound probe 18 includes at least one of an active matrix linear transducer and a phased array transducer including elevation focusing and beam steering capabilities. Because ultrasound probe 18 includes an active matrix linear transducer or a phased array transducer, the inherent spatial resolution is maintained over a much greater depth than with standard probes. Further, elevation focusing and carefully chosen compression paddle plastic materials, that enable the use of high frequency probes, high spatial resolution of the order of 250 microns for the ultrasound images is obtained with this system as validated on phantom and clinical images.

In one embodiment, a computer software program, installed on ultrasound imaging system 14, is used to drive ultrasound probe 18 in a pre-determined trajectory on compression paddle 56. The program also communicates with stepper controller 132 and the ultrasound system 14 to trigger the image and data acquisition and storage. In another embodiment, a computer software program, installed on tomosynthesis imaging system 20, is used to drive ultrasound probe 18 in a pre-determined trajectory on compression paddle 56. The program facilitates increasing ultrasound probe 18 positioning accuracy within approximately ±100 microns.

Additionally, imaging system 12 facilitates de-coupling the image acquisition process such that the hardware utilized for one examination, i.e., X-ray source 24 and detector 26, minimally affects the image quality of the other image generated using ultrasound probe 26. Further, system 12 facilitates a reduction in structured noise, cyst versus solid mass differentiation, and fill 3D visualization of multi-modality registered data sets in a single automated combined examination, thereby facilitating improved methods for localization and characterization of suspicious regions in breast images, thereby resulting in a reduction in unnecessary biopsies and a greater efficiency in breast scanning.

Since clinical ultrasound, and 3D, as well as 2D, digital X-rays are available in co-registered format using system 12, system 12 therefore provides a platform for additional advanced applications, such as, but not limited to, a multi-modality CAD algorithm, improved classification schemes for CAD. System 12 facilitates navigating breast biopsies with greater accuracy than available with 2D X-ray data sets because of the information in the depth dimension. Patients undergoing various forms of treatment for breast cancer may be monitored with system 12 to evaluate their response to therapy because of the automation of ultrasound scanning and therefore the reduced effect of variability in scanning. For example, using system 12, an X-ray and ultrasound image dataset may be acquired during an initial examination and a plurality of subsequent examinations occurring over various time intervals during treatment. During a subsequent examination, the patient may be positioned in a manner similar as positioned in the initial examination by using system 12 to image breast 22 ultrasonically with the same operating parameters as used when acquiring the first data set. Mutual information or feature based registration techniques may then be used to determine the x, y, and z displacements needed in iterative patient repositioning required to bring the two sets of ultrasound data into better registration with one another using clearly identifiable features on both data sets or other means. Such features could also be potentially implanted if surgical treatment is being used. This could provide the clinicians with data sets that are substantially registered with respect to each other since recurrent cancers are not uncommon, therefore system 12 may be used to track progress and modify the treatment regimen accordingly. Further, system 12 facilitates a reduced compression of breast 22 because of the mitigation of structured noise that is a major motivational factor for increased compression. Modifications to system 12 may also be made to enable the combination of stereo-mammography with 3D ultrasound.

While the invention has been described in terms of various specific embodiments, those skilled in the art will recognize that the invention can be practiced with modification within the spirit and scope of the claims.

What is claimed is:

1. A method for generating an image of an object of interest, said method comprising:
acquiring a first three-dimensional dataset of the object at a first position using an X-ray source and a detector;
acquiring a second three-dimensional dataset of the object at the first position using an ultrasound probe; and
combining the first three-dimensional dataset and the second three-dimensional dataset to generate a three-dimensional image of the object.

2. A method in accordance with claim 1 further comprising:
compressing an object of interest using a compression paddle;
positioning an ultrasound probe mover assembly adjacent the compression paddle such that the second three-dimensional dataset obtained with the ultrasound probe mover assembly is co-registered with the first three-dimensional dataset obtained through the compression paddle by mechanical design; and
coupling an ultrasound probe with the probe mover assembly such that the ultrasound probe emits an ultrasound output signal through the compression paddle and the object of interest.

3. A method in accordance with claim 1 further comprising registrating the first three-dimensional data set and the second three-dimensional data set during acquisition.

4. A method in accordance with claim 1 wherein combining the first three-dimensional dataset and the second three-dimensional dataset comprises registering the first three-dimensional dataset and the second three-dimensional dataset on a point-by-point basis.

5. A method in accordance with claim 1 wherein acquiring a second three-dimensional dataset of the object at the first position using an ultrasound probe comprises using an ultrasound probe including at least one of an active matrix linear transducer and a phased array transducer comprising elevation focusing and beam steering capabilities.

6. A method in accordance with claim 1 wherein acquiring a second three-dimensional dataset of the object at the first position using an ultrasound probe comprises using an ultrasound probe including a two-dimensional array of capacitive micro-machined ultrasonic transducers.

7. A method in accordance with claim 1 wherein positioning an ultrasound probe mover assembly adjacent the compression paddle comprises positioning an ultrasound probe mover assembly including an automated two-dimensional ultrasound probe mover assembly.

8. A method for generating an image of an object of interest, said method comprising:
compressing an object of interest using a compression paddle;
acquiring a first three-dimensional dataset of the object at a first position using an X-ray source and a detector;
positioning an ultrasound probe mover assembly adjacent the compression paddle such that the second three-dimensional dataset obtained with the ultrasound probe mover assembly is co-registered with the first three-dimensional dataset obtained through the compression paddle by mechanical design;
coupling an ultrasound probe with the probe mover assembly such that the ultrasound probe emits an ultrasound output signal through the compression paddle and the object of interest;
acquiring a second three-dimensional dataset of the object at the first position using an ultrasound probe; and
combining the first three-dimensional dataset and the second three-dimensional dataset to generate a three-dimensional image of the object.

9. A method for generating an image of an object of interest, said method comprising:
compressing an object of interest using a compression paddle;
acquiring a two-dimensional dataset of the object, at a first position, using an X-ray source and a detector;
positioning an ultrasound probe mover assembly adjacent the compression paddle such that the second three-dimensional dataset obtained with the ultrasound probe mover assembly is co-registered with the first three-dimensional dataset obtained through the compression paddle by mechanical design;
operationally coupling an ultrasound probe with the probe mover assembly such that the ultrasound probe emits an ultrasound output signal through the compression paddle and the object of interest;
acquiring a three-dimensional dataset of the object, at the first position, using an ultrasound probe; and
combining the two-dimensional dataset and the second three-dimensional dataset to generate a three-dimensional image of the object.

10. An apparatus comprising:
a compression paddle;
an ultrasound probe mover assembly mechanically aligned with said compression paddle; and
an ultrasound probe coupled with said probe mover assembly such that said ultrasound probe emits an ultrasound output signal through said compression paddle and said object of interest.

11. An apparatus in accordance with claim 10 wherein said paddle is coupled to a tomosynthesis imaging system.

12. An apparatus in accordance with claim 10 wherein said object of interest is a breast.

13. An apparatus in accordance with claim 10 wherein said ultrasound probe comprises at least one of an active matrix linear transducer and a phased array transducer.

14. An apparatus in accordance with claim 13 wherein at least one of said active matrix linear transducer and said phased array transducer comprises elevation focusing and beam steering capabilities.

15. An apparatus in accordance with claim 10 wherein a radiation source emits a radiation beam through said compression paddle and said object of interest to a detector assembly to generate a first three-dimensional dataset, said ultrasound probe emits an ultrasound output signal through said compression paddle and said object of interest to generate a second three-dimensional dataset.

16. An apparatus in accordance with claim 15 wherein a computer combines said first three-dimensional dataset and said second three-dimensional dataset to generate a co-registered three-dimensional dataset representative of said object of interest.

17. A medical imaging system for generating an image of an object of interest, said medical imaging system comprising:
a detector array;
at least one radiation source;
a compression paddle;
an ultrasound probe mover assembly mechanically aligned with said compression paddle;
an ultrasound probe coupled with said probe mover assembly such that said ultrasound probe emits an ultrasound output signal through said compression paddle and said object of interest; and
a computer coupled to said detector array, said radiation source, and said ultrasound probe, and configured to:
acquire a first three-dimensional dataset of the object at a first position using said X-ray source and said detector;
acquire a second three-dimensional dataset of the object at the first position using said ultrasound probe; and
combine the first three-dimensional dataset and the second three-dimensional dataset to generate a three-dimensional image of the object.

18. A medical imaging system in accordance with claim 17, wherein said computer further configured to physically co-register the first three-dimensional data set and the second three-dimensional data set during acquisition.

19. A medical imaging system in accordance with claim 17 wherein to combine the first three-dimensional dataset and the second three-dimensional dataset to generate a three-dimensional image, said computer further configured to register the first three-dimensional dataset and the second three-dimensional dataset on a point by point basis.

20. A medical imaging system for generating an image of an object of interest, said medical imaging system comprising:
a detector array;
at least one radiation source;
a compression paddle;
an ultrasound probe mover assembly mechanically aligned with said compression paddle;
an ultrasound probe coupled with said probe mover assembly such that said ultrasound probe emits an ultrasound output signal through said compression paddle and said object of interest; and
a computer coupled to said detector array, said radiation source, and said ultrasound probe, and configured to:
acquire a first three-dimensional dataset of the object at a first position using said X-ray source and said detector;
acquire a second three-dimensional dataset of the object co-registered with the first three-dimensional dataset, at the first position, using said ultrasound probe;
register the first three-dimensional dataset and the second three-dimensional dataset on a point by point basis; and
combine the first three-dimensional dataset and the second three-dimensional dataset to generate a three-dimensional image of the object.

21. A compression paddle comprising a plurality of composite layers, wherein said layers are sonolucent and radiolucent.

22. A compression paddle in accordance with claim 21 wherein said layers comprise at least one of a polycarbonate, a polymethylpentene, and a polystyrene, and combinations thereof.

23. A compression paddle in accordance with claim 21 wherein said layers comprise an ultrasonic attenuation less than approximately 3 dB at a plurality of ultrasound probe frequencies less than approximately 12 megahertz.

24. A compression paddle in accordance with claim 23 wherein said layers configured to optically transmit greater than approximately 80% of incident radiation.
